(19) United States
(12) Patent Application Publication
(10) Pub. No.: US 2024/0216263 A1
(43) Pub. Date: Jul. 4, 2024

(54) COMPOSITION FOR PREVENTING HAIR LOSS AND PROMOTING HAIR GROWTH COMPRISING EXTRACTS OF CORYLUS HETEROPHYLLA AND HELIANTHUS THBEROSUS AND METHOD FOR PREPARING SAME

(71) Applicant: Hyung-Ruel JU, Goyang-si, GG (KR)

(72) Inventor: Hyung-Ruel JU, Goyang-si, GG (KR)

(21) Appl. No.: 18/551,286

(22) PCT Filed: Mar. 19, 2021
(86) PCT No.: PCT/KR2021/003438

§ 371 Date: Feb. 19, 2024

(51) Int. Cl. A61K 8/9789; A61Q 5/00; A61Q 7/00

(52) U.S. Cl. CPC A61K 8/9789; A61Q 5/00; A61Q 7/00

(57) ABSTRACT

The present invention provides a composition affecting the scalp, having no side effect on the human body, and having an effect on hair loss prevention and hair growth, and a method for preparing same. More specifically, the present invention provides a composition for preventing hair loss and promoting hair growth and a method for preparing same, the composition comprising, as an active ingredient, a first mixture comprising extracts of Corylus heterophylla and Heilanthus tuberosus.

### TECHNICAL FIELD

The present disclosure relates to a composition for preventing hair loss and promoting hair growth including extracts of Corylus heterophylla and Helianthus tuberosus, and a method for manufacturing the same.

### BACKGROUND ART

It is a reality that the hair loss population continues to increase in all age groups due to various causes such as environmental factors, social factors, and the like, according to social development, and hair loss is increasing as the age group gradually decreases, regardless of gender. Hair loss symptoms not only give severe stress to anyone, but also cause loss of external and internal self-confidence. Hair loss is preferably diagnosed at an early stage and prevented or treated. In particular, when a cause of genetic hair loss and a cause of acquired hair loss are combined, symptoms of hair loss appear more severe, so it is good to diagnose and prevent the symptoms at an early stage.

Hair is formed in hair follicles, and hair follicles are composed of inner root sheath, outer root sheath, hair shaft, hair matrix, and the like, and dermal papilla cells are directly related to the ability to form hair follicles and perform a key role in forming hair follicles, so if there are no dermal papilla cells or they do not function properly, hair follicles cannot be formed.

Common causes of hair loss may include genetics, aging, excessive stress, dihydrotestosterone (DHT) production due to hypersecretion of male hormones, endocrine system diseases, drug use, sensitive scalp, seborrheic scalp, use of synthetic surfactants used almost every day, perm, dyeing, and the like. In addition, environmental pollution, improper eating habits and the Westernization of eating habits, drinking and smoking, lack of sleep, nutritional imbalance, excessive dieting, and the like are also cited as causes of hair loss.

In order to prevent such hair loss and improve advanced hair loss, a composition for preventing hair loss or improving hair growth including minoxidil has been studied as shown in Patent Registration No. 2003606, and a composition for preventing hair loss including finasteride has been studied as shown in Patent Registration No. 1921246, but minoxidil or finasteride have insignificant effect, vary greatly from person to person, and have side effects, so it is a reality that it difficult to use minoxidil or finasteride for a long period of time.

Therefore, there is an urgent need to develop an anti-hair loss agent, a hair tonic, a hair growth promoter, and a hair growth solution, that have no side effects and are safe.

### DETAILED DESCRIPTION OF INVENTION

An aspect of the present disclosure is to provide a composition acting on the scalp, having no side effects on the human body, and being effective in preventing hair loss and promoting hair growth, and a method for manufacturing the same.

According to an aspect of the present disclosure, the present disclosure is to provide a composition for preventing hair loss and promoting hair growth, the composition including an extract of Corylus heterophylla and an extract of Helianthus tuberosus.

According to an aspect of the present disclosure, the present disclosure is to provide a method for manufacturing a composition for preventing hair loss and promoting hair growth, the method including an operation of manufacturing a first mixture including hot water extracts of Corylus heterophylla and Helianthus tuberosus.

### Advantageous Effects of Invention

As set forth above, the composition for preventing hair loss and promoting hair growth of the present disclosure may reduce production of DHT hormone and cortisol hormone in the scalp, thereby providing effects of preventing hair loss, and promoting hair regrowth, hair growth, and hair growth promotion, thereby increasing the thickness and density of hair. In addition, since natural extracts are used, effects of preventing hair loss, and promoting hair regrowth, hair growth, and hair growth promotion may be provided without having side effects on the human body.

### BRIEF DESCRIPTION OF DRAWINGS

FIG. 1 illustrates a result of comparing a change in hair thickness and hair density in a scalp to which a composition for preventing hair loss and promoting hair growth was applied and a scalp to which the composition was not applied, according to an embodiment of the present disclosure.

FIG. 2 illustrates a result of comparing a change in hormones in the scalp when a composition for preventing hair loss and promoting hair growth was applied and when the composition is not applied, according to an embodiment of the present disclosure.

FIG. 3 illustrates a result of comparing a change in hormones in saliva when a composition for preventing hair loss and promoting hair growth was applied and when the composition is not applied, according to an embodiment of the present disclosure.

### BEST MODE FOR INVENTION

Hereinafter, preferred embodiments of the present disclosure will be described with reference to the accompanying drawings. However, the embodiments f the present disclosure may be modified in various forms, and the scope of the present disclosure is not limited to the embodiments described below.

Normal hair growth repeatedly generates and extinctions according to hair cycles, which are cycles of anagen in which hair grows due to active activity of hair follicles and cell division, catagen in which growth activity of hair follicles is suspended and rapidly atrophied, and telogen in which cell activity of hair follicles completely extinctions.

Meanwhile, testosterone is reduced to Dihydrotestosterone (DHT), which is highly active in hair follicles, by an action of 5α-reductase, and the reduced DHT binds to androgen receptors to delay protein synthesis in hair follicle cells, which shortens a growth period of hair follicles, inhibits division of hair matrix cells, and atrophies hair follicles to cause hair loss.

The 5α-reductase has two types (type 1 and type 2) of isoenzymes, and both are enzymes converting testosterone to DHT. Type 1 is mainly distributed in sebaceous glands, the epidermis, keratinocytes of hair follicles, dermal papilla cells, and sweat glands. Type 2 is mainly distributed in root sheaths of scalp hair follicles, epididymis, vas deferens, seminal vesicles, prostate, and fetal genital skin.

Therefore, there is a possibility of treating androgenic alopecia by inhibiting the isoenzymes of 5α-reductase. That is, in people with a congenital deficiency of type 2 5α-reductase, androgenetic alopecia does not occur or a forehead line recedes slightly backward. However, the activity of 5α-reductase is high in the hair follicles of patients with hair loss, leading to direct hair loss.

In addition, hair loss is influenced by the hormone cortisol. Cortisol is a hormone produced by the adrenal cortex that is involved in stress. A high cortisol level indicates a high stress state, and a low cortisol level indicates being less exposed to stressful situations. Under stress, the human body requires a lot of energy, and the hair reacts accordingly. Therefore, any hair loss is bound to be affected by stress, and under the influence of stress, the activity of Transforming growth factor-β1 (TGF-β1), which is a substance promoting transition from a growth phase to a catagen phase and a telogen phase during hair growth cycles, also increases.

That is, an increase in DHT and cortisol has a direct effect on hair loss, and when production of DHT and cortisol can be reduced, it will be possible to prevent hair loss or promote hair regrowth, hair growth, and hair growth promotion.

Accordingly, the present specification provides a composition having an excellent effect for preventing hair loss and promoting hair growth, by effectively reducing the production of DHT and cortisol having no side effects on the human body using natural substances.

Specifically, the present disclosure provides a composition for preventing hair loss and promoting hair growth including, as an active ingredient, a first mixture including extracts of Corylus heterophylla and Heilanthus tuberosus.

Furthermore, the present disclosure provides a composition for preventing hair loss and promoting hair growth, wherein the first mixture includes 1 to 3 parts by weight of the extract of Heilanthus tuberosus based on 1 part by weight of the extract of Corylus heterophylla.

The first mixture may use Corylus heterophylla including quercetin, which is used as an antioxidant and anti-aging substance by reducing skin irritation and inflammation, as a main ingredient, and Heilanthus tuberosus including a large amount of phenolic compounds, thereby having a strong antioxidant effect, and 15% or more of inulin, called natural insulin among vegetable substances.

Specifically, a main ingredient of Corylus heterophylla is a fatty acid, which is a quercetin-based glycoside. The quercetin is a kind of bioflavonoids found in plants and has anti-inflammatory and anti-allergic properties, has an antioxidant action to prevent oxidation of unsaturated fatty acids, and has an anti-aging effect. In addition, the quercetin purifies blood by lowering blood viscosity, reduces an amount of cholesterol in the blood, and helps prevent cardiovascular and cerebrovascular diseases and high blood pressure. In addition, tannin, one of the main ingredients of Corylus heterophylla, is a type of polyphenol and is useful for antioxidant, antibacterial, and anti-inflammatory effects. Corylus heterophylla also contains a natural anticancer substance called Taxol, which is effective in treating and preventing various cancers. In addition, Corylus heterophylla has an effect of increasing skin moisturization and elasticity. However, the effect of hair loss prevention and hair growth of Corylus heterophylla has not been known.

The extract of Corylus heterophylla may be extracted from leaves, stems or roots of hazel trees, and is preferably an extract using hazel fruits.

Furthermore, a root of Helianthus tuberosus is rich in vitamins, proteins, sugars, minerals, and potassium, such as vitamin B2, which makes immune function normal, vitamin C, which affects sugar metabolism, and niacin (vitamin B3). In addition, Helianthus tuberosus contains trace amount of fructose and glucose, and is rich in vegetable fiber, so it is effective for diet and constipation. Helianthus tuberosus contains a large amount of phenolic compounds, anthocyanin, carotenoid, and the like, and has strong antioxidant and anti-inflammatory effects. In particular, Helianthus tuberosus contains inulin, a low-calorie polysaccharide, which acts as a natural insulin that does not raise blood sugar levels. However, the effect of hair loss prevention and hair growth of the extract of Helianthus tuberosus has not been known.

Furthermore, the present disclosure provides a composition for preventing hair loss and promoting hair growth, in the active ingredient, the active ingredient further including a second mixture including at least one extract selected from a group consisting of Physalis peruviana, Chenopodium quinoa, Paeonia radix, and Pleuropterus multiflorus.

Preferably, the present disclosure provides a composition for preventing hair loss and promoting hair growth, wherein the second mixture includes 1 to 2 parts by weight of an extract of Chenopodium quinoa, an extract of Paeonia radix, and an extract of Pleuropterus multiflorus, respectively, based on 1 part by weight of the extract of Physalis peruviana.

The Physalis peruviana contains beta-sitosterol (β-sitosterol), which is phytosterol and provitamin D, and is widely used for arteriosclerosis, angina pectoris, myocardial infarction, cerebrovascular disease, and diabetes caused by cholesterol accumulation. Beta-sitosterol (β-sitosterol), is beneficial for wound healing, removal of inflammation, and helps with gum disease, tooth decay prevention, prostatic hyperplasia, hair loss, and the like.

Chenopodium quinoa is rich in betaine and has a high content of potassium, vitamin E, lysine, and saponin among grains. Lysine in Chenopodium quinoa is a kind of basic amino acid that helps in the production of antibodies, hormones, enzymes, collagen production and tissue formation and repair, and in particular, lysine is effective in promoting hair growth and hair elasticity, and a deficiency of lysine causes hair loss. Saponin in Chenopodium quinoa helps promote hair growth by improving blood circulation in the scalp, is involved in improving hair thickness, and inhibits skin aging and inflammation.

Paeonia radix has various efficacies and effects such as skin whitening and antioxidant effects. Furthermore, in the scalp and hair, it has an efficacy and effect of protecting hair from external damage, supplying moisture to the scalp to induce hair growth, and suppressing itching and dandruff. In addition, Paeonia radix inhibits the activity of type 2 5α-reductase and has excellent antioxidant and inhibits the activity of type 2 5α-reductase, a has hair growth effect, a hair loss prevention effect, an antioxidant, an anticancer effect, and the like.

In addition, Pleuropterus multiflorus has an effect of preventing hair loss, promoting hair growth, and inhibiting the activity of type 2 5α-reductase, and in particular, has an effect of promoting hair growth and blackening hair. In addition, Pleuropterus multiflorus activates the metabolic activity of cells in the body, especially scalp cells, to improve the condition of the scalp, thereby preventing hair loss.

The second mixture may be mixed with the first mixture and used, for example, in a volume ratio of 2:1 to 2:1, preferably 1:1. When a content of the second mixture is relatively significantly high in the volume ratio and exceeds the range of the present disclosure, a problem of reducing a scalp soothing effect may occur, and when a content of the first mixture is relatively significant and exceeds the scope of the present disclosure, problems may occur in moisturizing the scalp and hair.

Furthermore, the extract of the present disclosure may be a hot water extract of each raw material component. For example, the extract may be an extract extracted by soaking raw materials in water for a certain period of time and then applying heat, and the extracts of Corylus heterophylla and Helianthus tuberosus, and the extracts of Physalis peruviana, Chenopodium quinoa, Paeonia radix, and Pleuropterus multiflorus may all be used in a form of hot water extracts. For example, the extracts of Corylus heterophylla and Helianthus tuberosus may be obtained by soaking of Corylus heterophylla and Helianthus tuberosus in wafer for 5 hours and then heating the same at 80 to 120° C. for 8 to 22 hours.

Meanwhile, the composition for preventing hair loss and promoting hair growth of the present disclosure may be a cosmetic composition.

The cosmetic composition may be used in a form of being easily applied to the scalp, and may be used in any one formulation selected from a group consisting of, for example, a soap, shampoo, rinse, treatment, essence, tonic, cream, pack, lotion, nutrient, paste and gel, but the present disclosure is not limited thereto as long as it has a form that is easily applied to the scalp.

Furthermore, the present disclosure provides a method for manufacturing a composition for preventing hair loss and promoting hair growth.

In detail, the present disclosure provides a method for manufacturing a composition for preventing hair loss and promoting hair growth, including manufacturing a first mixture including hot water extracts of Corylus heterophylla and Helianthus tuberosus.

The operation of preparing the first solution may be performed by mixing the Corylus heterophylla and Helianthus tuberosus in a weight ratio of 1:1 to 3, and then mixing 1 to 5 times the weight of water based on a total weight of Corylus heterophylla and Helianthus tuberosus.

In this case, when the weight ratio of the Helianthus tuberosus to the Corylus heterophylla is outside of the above range, a problem in supplying desired nutrients may occur.

In addition, the present invention provides a method for manufacturing a composition for preventing hair loss and promoting hair growth, the method further including: an operation of manufacturing a second mixture including hot water extracts of Physalis peruviana, Chenopodium quinoa, Paeonia radix, and Pleuropterus multiflorus; and manufacturing a mixed extraction solution by mixing the second mixture with the first mixture.

The operation of manufacturing the second solution may be performed by mixing Chenopodium quinoa, Paeonia radix, and Pleuropterus multiflorus in an amount of 1 to 2 parts by weight, respectively, based on 1 part by weight of the Physalis peruviana, Chenopodium quinoa, Paeonia radix, and Pleuropterus multiflorus, and then mixing 1 to 5 times the weight of water with a total weight of the Physalis peruviana, Chenopodium quinoa, Paeonia radix, and Pleuropterus multiflorus.

In this case, based on the Physalis peruviana, when a weight ratio of Chenopodium quinoa, Paeonia radix, and Pleuropterus multiflorus is outside of the above range, a difference in the extracted components may occur, which may cause problems in supplying nutrients.

The hot water extraction may be performed by heating at a temperature of 80 to 120° C. for 8 to 22 hours, and for example, may be performed by heating an aqueous mixture of Helianthus tuberosus, Corylus heterophylla, and water at a temperature of 100° C. for 16 hours.

However, in the present disclosure, for efficient extraction of components, the first solution and the second solution may be soaked in water for 5 to 8 hours before heating.

Furthermore, the present disclosure may further include an operation of putting a first mixture, a second mixture, or a mixed extraction solution in a concentrator and concentrating the same at 80 to 90° C. for 5 to 15 hours before or after the operation of preparing the mixed extraction solution. For example, the mixed extraction solution may be concentrated at 80 to 90° C. for 5 to 15 hours.

Impurities of the mixed extraction solution concentrated as described above may be removed through filtration, and stabilized through aging.

For example, the filtration may be performed with a 250 to 350 mesh filtering device, and almost all impurities in the concentrated mixed extraction solution may be removed through the filtering device. In addition, the aging may be performed at 20 to 25° C. for 60 to 80 hours, through which the mixed extraction solution may be stabilized.

Hereinafter, the present disclosure will be described in more detail through specific examples. The following examples are merely examples to aid understanding of the present disclosure, and the scope of the present disclosure is not limited thereto.

### MODE FOR INVENTION

### Preparation Example

Corylus heterophylla (fruits of hazel trees), Helianthus tuberosus, Paeonia radix, and Pleuropterus multiflorus were washed and dried, and cut into pieces, and

Physalis peruviana and Chenopodium quinoa were washed and used, and water was used as an extraction solvent.

More specifically, in the extraction, an aqueous solution was prepared by mixing 50 g of the Corylus heterophylla, 100 g of the Helianthus tuberosus, and 750 g of water, the aqueous solution was heated at 100° C. for 16 hours and then completely cooled at room temperature (20° ° C. to 25° C.), to prepare a first mixture.

Furthermore, an aqueous solution was prepared by mixing 100 g of the Physalis peruviana, 150 g of the Chenopodium quinoa, 150 g of the Paeonia radix, 150 g of the Pleuropterus multiflorus, and 2,750 g of water, the aqueous solution was heated at 100° ° C. for 16 hours, and then completely cooled at room temperature (20° ° C. to 25° C.), to prepare a second mixture.

The first mixture and the second mixture were mixed in a volume ratio of 1:1, concentrated at 85° ° C. for 10 hours, and then completely cooled at room temperature (20° ° C. to 25° C.) to obtain a mixed extraction solution.

After filtering the mixed extraction solution through a 300-mesh filter, the obtained composition was aged at room temperature (20° ° C. to 25° C.) for 72 hours to obtain a composition for preventing hair loss and promoting hair growth.

The composition for preventing hair loss and promoting hair growth obtained through this process was named JU7505, prepared as a solid shampoo (JU7505), and applied clinically for preventing hair loss and promoting hair growth. Example 1 and Comparative Example 1 below describe the clinical study, and this clinical study was conducted at Seoul National University Bundang Hospital.

### Example 1

A patient with hair loss was treated with a solid shampoo prepared from the composition for preventing hair loss and promoting hair growth prepared in Preparation Example, and hair thickness, hair density, and various hormone levels were measured.

### Comparative Example 1

Hair thickness, hair density, and various hormone levels were measured without any treatment of patients with hair loss.

For a specific experiment, Example 1 and Comparative Example 1 were performed on ordinary people suffering from hair loss as shown in Table 1 below. For comparison, normal people (ordinary people without hair loss) were set as a control.

TABLE 1

| | | | CONTROL (NORMAL PEOPLE, |
|---|---|---|---|
| | COMPARATIVE | | ORDINARY PEOPLE |
| | EXAMPLE 1 | EXAMPLE 1 | WITHOUT HAIR LOSS) |
| AGE AVERAGE (RANGE) | 47.7(30-61) | 49.0(29-65) | 41.24(22-63) |
| HEIGHT (cm) | 172.6(165-183) | 172.1(160-192) | 174.8(161-186) |
| WEIGHT (kg) | 75.6(55-92) | 73.5(55-117) | 79.3(63-110) |
| SMOKING HISTORY | | | |
| 1.NON-SMOKING | 1: 23.6%: | 1: 21%; | 1: 35%; |
| 2.HAVING A HISTORY OF SMOKING | 2: 16.4%; | 2: 35.5%; | 2: 15%; |
| BUT QUITTING SMOKING | | | |
| 3.DURING SMOKING | 3: 60% | 3: 43.5% | 3: 50% |
| NON-SMOKING PERIOD FOR | 19.1(8-30) | 16.6(2-30) | 9.64(5-20) |
| NON-SMOKERS (YEARS) | | | |
| AMOUNT OF SMOKING BY | 13.9(10-20) | 13.4(2-30) | 16.5(7-30) |
| SMOKERS BEFORE NON-SMOKING | | | |
| PERIOD FOR NON-SMOKERS | | | |
| (AMOUNT OF CIGARETTES/DAY) | | | |
| SMOKING PERIOD FOR | 23.9(10-40) | 22.5(6-36) | 17.1(3-40) |
| SMOKERS (YEARS) | | | |
| AMOUNT OF SMOKING BY | 16.0(2-40) | 17.4(10-40) | 16.4(2-40) |
| SMOKERS (AMOUNT OF | | | |
| CIGARETTES/DAY) | | | |
| DRINKING DAYS (DAY/WEEK) | 1.4(0-6) | 1.7(0-7) | 1.7(0-7) |
| AMOUNT OF DRINKING | 5.7(0-20) | 6.7(0-24) | 7.6(0-30) |
| (GLASS/WEEK) | | | |
| WHETHER THERE ARE | PEOPLE IN THE FAMILY | PEOPLE IN THE FAMILY | PEOPLE IN THE FAMILY |
| PEOPLE HAVING HAIR LOSS | HAVE HAIR LOSS: 65.5%; | HAVE HAIR LOSS: 65.6%; | HAVE HAIR LOSS: 33%; |
| IN FAMILY | NO PEOPLE IN FAMILY | NO PEOPLE IN FAMILY | NO PEOPLE IN FAMILY |
| | HAVE HAIR LOSS: 34.5% | HAVE HAIR LOSS: 34.4% | HAVE HAIR LOSS: 67% |
| PEOPLE HAVING HAIR LOSS | PATRILINEAL LINE: 92.8%; | PATRILINEAL LINE: 93.1%; | PATRILINEAL LINE: 90%; |
| (MARKED AS DUPLICATE) | MATRILINEAL LINE: 14.3%; | MATRILINEAL LINE: 6.9%; | MATRILINEAL LINE: 10%; |
| | SIBLING: 32.1%, | SIBLING: 24.1%, | SIBLING: 20%, |
| | PATERNAL | PATERNAL | PATERNAL |
| | GRANDPARENTS: 14.3%; | GRANDPARENTS: 17.4%; | GRANDPARENTS: 15%; |
| | MATERNAL | MATERNAL | MATERNAL |
| | GRANDPARENTS: 2.5% | GRANDPARENTS: 3.4% | GRANDPARENTS: 2.5% |
| HAIR LOSS PERIOD (YEARS) | 9.2(1-30) | 10.6(2-26) | |
| HAIR LOSS FORM | O + MC TYPE: 36.3% | O + MC TYPE: 36.7% | |
| | C TYPE: 7.3% | C TYPE: 13.3% | |
| | O TYPE: 30.9% | O TYPE: 30% | |
| | M TYPE: 12.7% | M TYPE: 20% | |

### [Measurement of Hair Thickness and Hair Density]

In the Example 1, Comparative Example 1, and a head of a control, a virtual line (glabellar line) leading to a crown from a midpoint between eyebrows and a virtual line (lateral line) extending from an apex of a left auricle to a right auricle were set as a reference line, so a measurement point was determined.

About 50 mg of hair (about half the thickness of a matchstick when tied in a row) was collected from the scalp at a point within a radius of 2 cm based on each individual's measurement point, using thinning scissors, and hair thickness of the collected hair was measured using a thickness gauge.

After hair collection, using a hair analyzer equipped with a 50× or 250× magnification lens, a photograph of each individual's scalp at the measurement point was taken, and hair density was measured by counting the number of hairs included in one angle of view of the photograph.

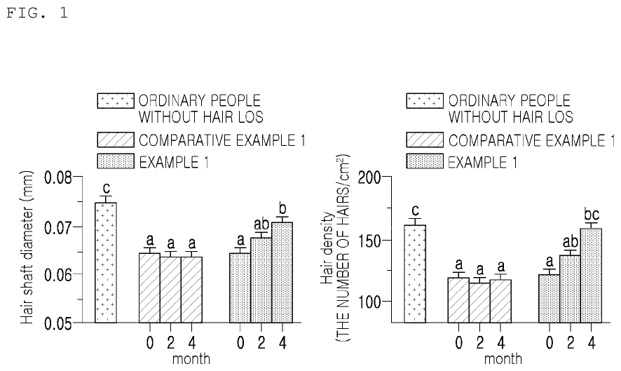

In the above method, hair thickness and hair density were repeatedly measured before using a solid shampoo (after 0 month), after 2 months and after 4 months of using the solid shampoo, and the change thereof was observed, and the results of measuring the hair thickness and hair density of Example 1 and Comparative Example 1 were shown in Table 2 and FIG. 1 below.

TABLE 2
HAIR THICKNESS(DIAMETER, mm)

| | | EXAMPLE 1 | | | COMPARATIVE EXAMPLE 1 | | |
|---|---|---|---|---|---|---|---|
| | CONTROL | AFTER | AFTER | AFTER | AFTER | AFTER | AFTER |
| | — | 0 MONTH | 2 MONTIIS | 4 MONTHS | 0 MONTH | 2 MONTHS | 4 MONTIIS |
| MEAN | 0.07495 | 0.06376 | 0.06768 | 0.07085 | 0.06444 | 0.06371 | 0.06376 |
| STANDARD | 0.009935 | 0.0092 | 0.009072 | 0.006944 | 0.008626 | 0.008745 | 0.008093 |
| DEVIATION, | | | | | | | |
| SD | | | | | | | |
| STANDARD | 0.001328 | 0.001168 | 0.001191 | 0.001157 | 0.001153 | 0.001179 | 0.001133 |
| ERROR OF | | | | | | | |
| THE MEAN, | | | | | | | |
| SEM) | | | | | | | |

HAIR DENSITY (THE NUMBER OF HAIRS/cm²)

| | | EXAMPLE 1 | | | COMPARATIVE EXAMPLE 1 | | |
|---|---|---|---|---|---|---|---|
| | | AFTER | AFTER | AFTER | AFTER | AFTER | AFTER |
| | CONTROL | 0 MONTH | 2 MONTHS | 4 MONTHS | 0 MONTH | 2 MONTHS | 4 MONTHS |
| MEAN | 161.9 | 122 | 136.9 | 159.4 | 119.1 | 116 | 116.9 |
| STANDARD | 24.86 | 44.26 | 41.07 | 28 | 45.18 | 45.94 | 42.39 |
| DEVIATION, | | | | | | | |
| SD | | | | | | | |
| STANDARD | 3.589 | 5.666 | 5.696 | 4.875 | 6.206 | 7.657 | 7.065 |
| ERROR OF | | | | | | | |
| THE MEAN, | | | | | | | |
| SEM) | | | | | | | |

As shown in Table 2 and FIG. 1, it was confirmed that hair thickness and the number of hairs increased when the composition of the present disclosure was treated with ordinary people suffering from hair loss.

### [Measurement of Changes in Hormone Production in the Scalp]

Changes in hormone production in the scalp were performed in the following sequence.

### 1) Sample Purification

From each participant, 80-100 mg of hair with a length of about 2 cm was collected from an edge area where hair loss is in progress. A weight of the collected hair was again quantified using a chemical balance, and these hairs were finely cut at 3-4 mm intervals using surgical scissors and tweezers.

### 2) Quantitative Analysis of DAT

Steroid hormones contained in the collected hair were extracted using the method of Deng et al. (1999).

Specifically, the finely cut hair was alkaline decomposed at room temperature using 1 ml of 1 N NaOH, and neutralized to pH 7.0 using 1 N HCl after 1 hour. Steroid hormones contained in the neutralized solution were extracted using 4 ml of 100% diethyl ether. Steroid hormones contained in the diethyl ether were concentrated using a centrifugal evaporator, and 1 ml of phosphate buffer solution for hormone analysis was added and stored in a freezer at −80° C. This was set as hair steroid solution-1 (HSS-1).

Since a molecular structure of testosterone and dihydrotestosterone (DHT) are mutually similar, when trying to quantitatively analyze hormones using radioimmunoassay, an antibody of DHT cannot distinguish between testosterone and DHT. Therefore, after oxidizing and removing testosterone using the method of Werawatgoompa et al. (1982), DHT was quantitatively analyzed. Specifically, 500 ul of potassium permanganate (500 mg/100 ml) was added to 0.5 ml of HSS-1, and then left at room temperature for 20 minutes to remove testosterone contained in HSS-1, and 2 ml of 100% diethyl ether was added to the mixture and stirred for 30 minutes to extract other steroid hormones contained in the mixture. Steroid hormones contained in diethyl ether were concentrated using a centrifugal evaporator, and 0.2 ml of phosphate buffer solution for hormone analysis was added for DHT analysis, and then stored in a freezer at −80° C. until right before hormone analysis.

### 3) Analysis of Hormones Contained in Hair

Steroid hormones contained in HSS-1 were analyzed using radioimmunoassay. Cortisol, testosterone, dehydroepiandrosterone (DHEA) and epitestosterone (Epi-T) were analyzed using HSS-1. An antibody to cortisol was purchased from USbiological life sciences (MA, USA) and used, an antibody to DHEA was purchased from LSBio (WA, USA) and used, an antibody to testosterone was purchased from Absolue Antibody (MA, USA) and used, an antibody to Epi-T was purchased from Creative Diagnostics (NY, USA) and used. Standard materials each for hormone required for quantitative hormone analysis were purchased from Sigma-aldrich (MO, USA) and used, ¹²⁵I was bound to steroid hormones using the Chloramine-T method, and (steroid-¹²⁵I) was used for quantitative hormone analysis.

Steroid hormone analysis using radioimmunoassay followed the method of Ahn et al. (2007). Specifically, a total amount of radioactivity contained in each test tube was measured using a gamma counter (PerkinElmer life sciences, MA, USA), and two or three standard samples were usually applied to each analysis process, and each sample was subjected to two repetitive experimental processes. Steroid hormone concentrations in test tubes after radiation measurement were calculated using a Riasmart program (PerkinElmer life sciences, MA, USA) on a personal computer. Intra- and inter-experiment coefficients of variation (CV) in a steroid analysis process using radioimmunoassay were less than 10% and 5%, respectively, and maximum sensitivity to analyze corticosterone was 2.5 pg/tube.

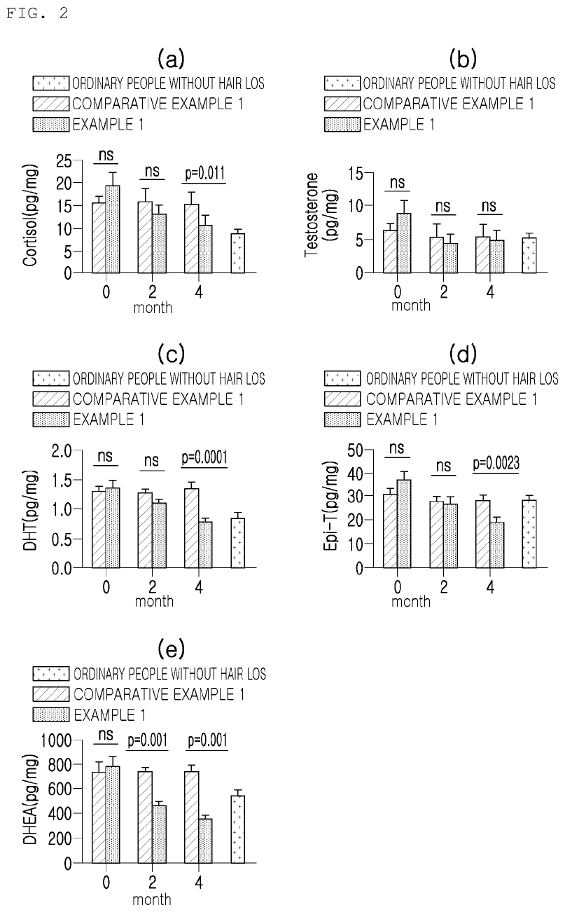

Using the same method as above, the results of measuring changes in hormone production in the scalp of the control, Example 1 and Comparative Example 1 were shown in Table 3 and FIG. 2 below.

TABLE 3

| | | | EXAMPLE 1 | | | COMPARATIVE EXAMPLE 1 | | |
|---|---|---|---|---|---|---|---|---|
| | | CONTROL | AFTER | AFTER | AFTER | AFTER | AFTER | AFTER |
| | | — | 0 MONTH | 2 MONTHS | 4 MONTHS | 0 MONTH | 2 MONTHS | 4 MONTHS |
| CORTISOL | MEAN | 8.929 | 19.4 | 13.35 | 10.69 | 15.55 | 15.98 | 15.33 |
| (pg/mg) | STANDARD | 1.032 | 2.949 | 1.763 | 2.254 | 1.879 | 2.953 | 2.722 |
| | ERROR OF | | | | | | | |
| | THE MEAN, | | | | | | | |
| | SEM | | | | | | | |
| TESTOSTERONE | MEAN | 5.137 | 8.832 | 4.485 | 4.869 | 6.306 | 5.285 | 5.309 |
| (pg/mg) | STANDARD | 0.6254 | 1.155 | 0.237 | 0.308 | 0.8011 | 0.3009 | 0.3056 |
| | ERROR OF | | | | | | | |
| | THE MEAN, | | | | | | | |
| | SEM | | | | | | | |
| DHT (pg/mg) | MEAN | 0.8571 | 1.354 | 1.1 | 0.7965 | 1.304 | 1.263 | 1.347 |
| | STANDARD | 0.1008 | 0.1261 | 0.07082 | 0.05432 | 0.07467 | 0.07069 | 0.09401 |
| | ERROR OF | | | | | | | |
| | THE MEAN, | | | | | | | |
| | SEM | | | | | | | |
| Epi-T (pg/mg) | MEAN | 28.29 | 37.61 | 27.19 | 19.07 | 30.99 | 27.83 | 28.33 |
| | STANDARD | 2.702 | 3.017 | 2.846 | 2.539 | 2.675 | 2.206 | 2.302 |
| | ERROR OF | | | | | | | |
| | THE MEAN, | | | | | | | |
| | SEM | | | | | | | |
| DHEA (pg/mg) | MEAN | 552.7 | 787.8 | 459.1 | 364.5 | 738.3 | 738.9 | 744.4 |
| | STANDARD | 43.61 | 82.16 | 24.96 | 23.88 | 84.45 | 49.87 | 54.49 |
| | ERROR OF | | | | | | | |
| | THE MEAN, | | | | | | | |
| | SEM | | | | | | | |

As shown in Table 3 and FIG. 2, it was confirmed that cortisol, testosterone, DHT, Epi-T, and DHEA in the scalp were all reduced when the composition of the present disclosure was treated with ordinary people suffering from hair loss. Therefore, it was confirmed that the composition of the present disclosure affects a change in hormones in the scalp and hair follicles.

### [Measurement of Changes in Hormone Production in Saliva]

Measurement of a change in hormones in saliva was performed in the following order.

### 1) Sample Purification

Saliva samples obtained from each participant went through a primary purification process. Specifically, the saliva sample was centrifuged at 2000×g for 30 minutes to remove foreign substances contained in saliva, and after centrifugation, a supernatant was taken and left at −80° C. for 2 hours, then rapidly thawed and centrifuged at high speed (at 15000×g for 30 minutes), and then, the supernatant was stored at −80° C. until immediately before hormone analysis. This was designated as sample 1.

### 2) Preparation for Quantitative Analysis of DHT

After taking a sample of 300 ul from the sample, 0.3 ml of potassium permanganate (500 mg/100 ml) was added to the sample, and left at room temperature for 20 minutes to remove testosterone contained in HSS-1. 1.2 ml of 100% diethyl ether was added to the mixed solution and stirred for 30 minutes to extract other steroid hormones contained in the mixed solution.

Furthermore, a steroid hormone contained in diethyl ether was concentrated using a centrifugal evaporator, and then 0.2 ml of phosphate buffer solution for hormone analysis was added to analyze DHT, and then stored in a freezer at −80° C. until immediately before hormone analysis. This was designated as sample 2. Furthermore, a concentration of DHT was quantitatively analyzed from the sample 2 in the same manner as in [Measurement of changes in hormone production in the scalp].

### 1) Analysis of Hormones Contained in Saliva

A steroid hormone contained in the specimen was quantitatively analyzed by radioimmunoassay based on the method of Ahn et al. (Ahn et al., 2007). Since a concentration of steroid hormones contained in sample 1 is tens of times lower than that of blood (20-30 times in the case of cortisol), commercially available hormone analysis kits are not suitable for measuring steroid hormones in saliva.

Therefore, the concentration of steroid hormones contained in saliva was measured using a liquid phased-double antibody radioimmunoassay. Cortisol, testosterone, dehydroepiandrosterone (DHEA) and epitestosterone (Epi-T) were analyzed. The antibody to cortisol was purchased from US biological life sciences (MA, USA) and used, the antibody to DHEA was purchased from LSBio (WA, USA) and used, the antibody to testosterone was purchased from Absolue Antibody (MA, USA) and used, and the antibody to Epi-T was purchased from Creative Diagnostics (NY, USA) and used. A standard material for each hormone required for quantitative hormone analysis were purchased from Sigma-aldrich (MO, USA) and used. ¹²⁵I was bound to steroid hormones using a Chloramine-I method (steroid-¹²⁵I) and used for quantitative hormone analysis.

In the measurement process, standard samples and 0.1 ml of the sample of a predetermined concentration are put into a measurement tube, respectively, GPBS was added instead of the antibody to hormones in a test tube to which steroid hormones were not added, in order to measure non-specific binding, and charcoal-stripped saliva was added to a test tube including standard samples of steroid hormones. A total radiation dose of the ¹²⁵I-labeled steroid hormone was approximately 50,000 cpm/tube, and the antibody was diluted to 30-35% binding. A total volume of a primary reaction was 0.4 ml per a test tube, and a standard sample and analysis targets were reacted at 4° C. for 24 hours. For a secondary reaction the next day, after additional reaction at room temperature for 30 minutes, 0.1 ml of a secondary antibody was added to each test tube and then reacted for another 30 minutes. After the reaction was completed, 0.5 ml of 10% PEG was added to each test tube, reacted for 15 minutes, and centrifuged at 4° C. at 1500 g for 20 minutes. After centrifugation, a supernatant was removed using a vacuum pump, and a radiation dose of precipitates remaining in each test tube was measured using a gamma ray meter (Cobra 5005, PerkinElmer Life and Analytical Sciences). A concentration of each measured hormone was calculated from a standard curve using Riasmart software (PerkinElmer Life and Analytical Sciences).

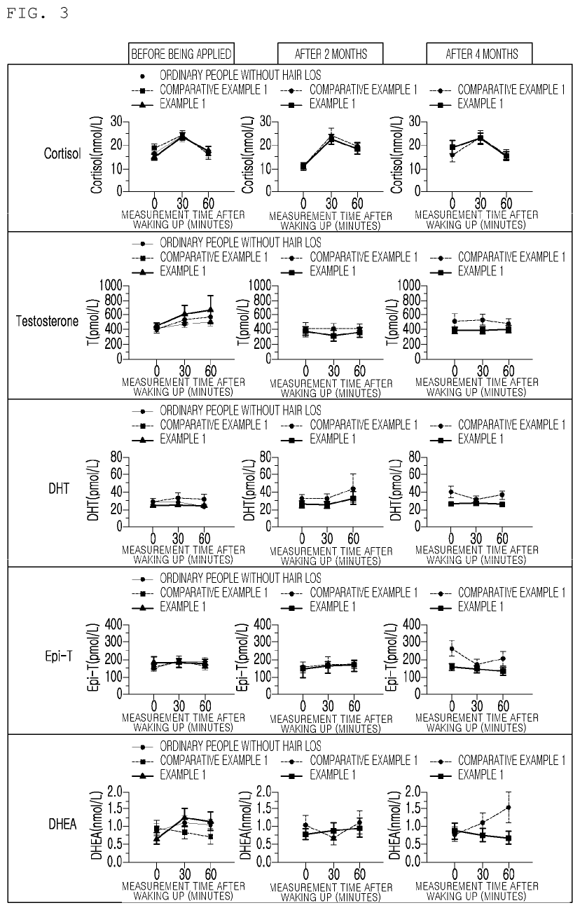

Results of measuring changes in hormone production in the control, and saliva of Example 1, and Comparative Example 1 by the same method as described above are shown in Table 4 and FIG. 3 below.

TABLE 4

| | | MEASURE- | | | | | | | |
|---|---|---|---|---|---|---|---|---|---|
| | | MENT | | EXAMPLE 1 | | | COMPARATIVE EXAMPLE 1 | | |
| | | TIME AFTER | | BEFORE | | | BEFORE | | |
| | | WAKING UP | CONTROL | BEING | AFTER | AFTER | BEING | AFTER | AFTER |
| | | (MINUTES) | — | APPLIED | 2 MONTHS | 1 MONTHS | APPLIED | 2 MONTHS | 4 MONTHS |
| CORTISOL | AVERAGE | 0 | 15.98 | 14.8 | 11.2 | 19.51 | 18.64 | 10.31 | 15.95 |
| (nmol/L) | (STANDARD | | (1.452) | (1.156) | (1.376) | (2.839) | (1.884) | (1.286) | (2.832) |
| | ERROR) | 30 | 23.74 | 23.93 | 22.37 | 22.99 | 24.17 | 24.35 | 23.54 |
| | | | (1.666) | (1.411) | (2.264) | (2.379) | (1.995) | (2.744) | (2.793) |
| | | 60 | 16.03 | 17.65 | 18.7 | 15.81 | 15.88 | 19.25 | 16.01 |
| | | | (1.146) | (1.759) | (2.507) | (1.6) | (1.921) | (1.364) | (2.294) |
| TES- | AVERAGE | 0 | 416.3 | 451.2 | 384.8 | 391.5 | 411.6 | 414.9 | 528.9 |
| TOSTERONE | (STANDARD | | (37.77) | (40.86) | (60.69) | (34.49) | (35.01) | (95.7) | (109.6) |
| (pmol/L) | ERROR) | 30 | 483 | 618.7 | 322.7 | 407.2 | 537.5 | 428.1 | 546.8 |
| | | | (48.97) | (119.8) | (71.88) | (38.44) | (76.25) | (69.15) | (81.33) |
| | | 60 | 516.1 | 683 | 368.9 | 409.7 | 577.6 | 426.9 | 499.7 |
| | | | (65.29) | (188.5) | (69.86) | (44.17) | (96.41) | (63.96) | (65.43) |
| DHT | AVERAGE | 0 | 28.43 | 24.28 | 27.06 | 27.62 | 29.2 | 33.18 | 41.17 |
| (pmol/L) | (STANDARD | | (2.108) | (1.723) | (3.065) | (1.75) | (2.688) | (4.288) | (6.641) |
| | ERROR) | 30 | 28.96 | 25.25 | 26.4 | 28.71 | 33.31 | 33.01 | 32.69 |
| | | | (3.354) | (2.141) | (4.027) | (2.187) | (5.638) | (5.316) | (3.505) |
| | | 60 | 23.63 | 24.42 | 33.83 | 27.43 | 31.15 | 44.86 | 37.49 |
| | | | (1.592) | (2.241) | (7.451) | (2.55) | (5.989) | (16.53) | (4.788) |
| Epi-T | AVERAGE | 0 | 156.4 | 183.7 | 155.6 | 165.3 | 155.8 | 166.7 | 271.1 |
| (pmol/L) | (STANDARD) | | (19.29) | (34.19) | (11.94) | (15.99) | (14.66) | (29.27) | (47.42) |
| | ERROR) | 30 | 187.4 | 181.1 | 171.1 | 151.8 | 188.1 | 178.3 | 178.3 |
| | | | (29.28) | (24.24) | (26.13) | (17.97) | (31.68) | (49.99) | (31.86) |
| | | 60 | 186.8 | 179.4 | 181.8 | 138.8 | 165.2 | 178.9 | 210.8 |
| | | | (30.87) | (24.83) | (26.08) | (22.38) | (23.15) | (22.1) | (40.65) |
| DHEA | AVERAGE | 0 | 0.9091 | 0.6574 | 0.7774 | 0.85 | 0.9518 | 1.016 | 0.7533 |
| (nmol/L) | (STANDARD | | (0.1768) | (0.1559) | (0.1596) | (0.224) | (0.2152) | (0.2753) | (0.1899) |
| | ERROR) | 30 | 1.1 | 1.248 | 0.8532 | 0.7336 | 0.8355 | 0.6638 | 1.055 |
| | | | (0.1959) | (0.252) | (0.2319) | (0.1894) | (0.1816) | (0.1992) | (0.2875) |
| | | 60 | 1.043 | 1.142 | 0.9579 | 0.6536 | 0.7136 | 1.094 | 1.5 |
| | | | (0.2444) | (0.2637) | (0.2579) | (0.1859) | (0.2004) | (0.3184) | (0.4343) |

As shown in Table 4 and FIG. 3, when the composition of the present disclosure was treated with the general public suffering from hair loss, it was confirmed that saliva's cortisol, testosterone, DHT, Epi-T and DHEA in saliva were not significantly different (no significant difference) in hormones from those not treated with the composition of the present disclosure.

Therefore, it was confirmed that the composition of the present disclosure significantly lowers the production of hormones in the scalp, but does not cause a significant change in the production of hormones in the body, and thus has no side effects on the human body.

While example embodiments have been shown and described above, it will be apparent to those skilled in the art that modifications and variations could be made without departing from the scope of the present disclosure as defined by the appended claims.

1. A composition for preventing hair loss and promoting hair growth, comprising, as an active ingredient,
a first mixture including an extract of Corylus heterophylla and an extract of Heilanthus tuberosus.

2. The composition for preventing hair loss and promoting hair growth of claim 1, wherein the first mixture comprises 1 to 3 parts by weight of an extract of Heilanthus tuberosus, based on 1 part by weight of the extract of Corylus heterophylla.

3. The composition for preventing hair loss and promoting hair growth of claim 1, wherein the active ingredient further comprises a second mixture including at least one extract selected from a group consisting of Physalis peruviana, Chenopodium quinoa, Paeonia radix, and Pleuropterus multiflorus.

4. The composition for preventing hair loss and promoting hair growth of claim 3, wherein the second mixture comprises 1 to 2 parts by weight of each of an extract of Chenopodium quinoa, an extract of Paeonia radix, and an extract of Pleuropterus multiflorus, based on 1 part by weight of the extract of Physalis peruviana.

5. The composition for preventing hair loss and promoting hair growth of claim 3, wherein the second mixture is mixed with the first mixture in a volume ratio of 2:1 to 1:2.

6. The composition for preventing hair loss and promoting hair growth of claim 1, wherein the extract is a hot water extract of each raw material component.

7. The composition for preventing hair loss and promoting hair growth of claim 1, wherein the composition for preventing hair loss and promoting hair growth is a cosmetic composition.

8. The composition for preventing hair loss and promoting hair growth of claim 7, wherein the cosmetic composition is any one formulation selected from a group consisting of soap, shampoo, rinse, treatment, essence, tonic, cream, pack, lotion, nutrient, paste, and gel.

9. A method for manufacturing a composition for preventing hair loss and promoting hair growth, comprising:
manufacturing a first mixture including hot-water extracts of Corylus heterophylla and Heilanthus tuberosus.

10. The method for manufacturing a composition for preventing hair loss and promoting hair growth of claim 9, further comprising:
manufacturing a second mixture including hot-water extracts of Physalis peruviana, Chenopodium quinoa, Paeonia radix, and Pleuropterus multiflorus; and
manufacturing a mixed extraction solution by mixing the second mixture with the first mixture.

11. The method for manufacturing a composition for preventing hair loss and promoting hair growth of claim 9, wherein the operation of manufacturing the first mixture is performed by mixing the Corylus heterophylla and Heilanthus tuberosus in a weight ratio of 1:1 to 3, and then mixing 1 to 5 times the weight of water based on a total weight of the Corylus heterophylla and Heilanthus tuberosus.

12. The method for manufacturing a composition for preventing hair loss and promoting hair growth of claim 10, wherein the operation of manufacturing the second mixture is performed by mixing the Physalis peruviana, Chenopodium quinoa, Paeonia radix, and Pleuropterus multiflorus in a content of 1 to 2 parts by weight, respectively, based on 1 part by weight of the Physalis peruviana, and then mixing 1 to 5 times the weight of water with a total weight of the Physalis peruviana, Chenopodium quinoa, Paeonia radix, and Pleuropterus multiflorus.

13. The method for manufacturing a composition for preventing hair loss and promoting hair growth of claim 10, wherein the hot water extraction is performed by being heated at a temperature of 80 to 120° C. for 8 to 22 hours.

14. The method for manufacturing a composition for preventing hair loss and promoting hair growth of claim 10, further comprising:
concentrating a first mixture, a second mixture, or a mixed extraction solution in a concentrator at 80 to 90° C. for 5 to 15 hours before or after the operation of preparing the mixed extraction solution.

15. The method for manufacturing a composition for preventing hair loss and promoting hair growth of claim 14, further comprising:
aging after filtering the concentrated mixed extraction solution.

16. The method for manufacturing a composition for preventing hair loss and promoting hair growth of claim 15, wherein the filtration is performed with a filtering device of 250 to 350 mesh.

17. The method for manufacturing a composition for preventing hair loss and promoting hair growth of claim 15, wherein the aging is performed at 20 to 25° C. for 60 to 80 hours.
